(19) United States
(12) Patent Application Publication
(10) Pub. No.: US 2012/0156154 A1
(43) Pub. Date: Jun. 21, 2012

(54) Preservative and Embalming Method and System

(76) Inventors: Paul Ash Minturn, La Jolla, CA (US); Lowell D. Haugen, Gig Harbor, WA (US)

(21) Appl. No.: 13/333,528

(22) Filed: Dec. 21, 2011

Related U.S. Application Data
(60) Provisional application No. 61/425,321, filed on Dec. 21, 2010.

(51) Int. Cl. A01N 59/16; A01P 3/00; A01N 1/00; A01P 1/00; B82Y 99/00; B82Y 5/00

(52) U.S. Cl. 424/75; 424/618; 977/773; 977/904

(57) ABSTRACT

Unique methods for preparing and producing non-toxic, liquid customized preservative substance formulations that may be used as embalming, sterilizing, disinfectant, preservative, mold destroying and fungus extermination solutions. These substances can: (a) safely resist or retard natural destructive processes; (b) save or keep things from decaying, decomposition, deterioration, spoiling, putrefication or fermentation; (c) continually prevent or inhibit unwanted microbial growth; and, (d) be used as embalming medical devices to significantly retard the carious destructive processes of nature with anti-microbial properties. These formulations forestall, prevent and inhibit undesirable chemical/biological changes, microbial/bacterial actions, plus retard decomposition or other negative changes. The formulations are comprised of ultra-fine minute nano-sized colloidal silver particles in pure distilled water resulting in stable suspended preparations. The formulations can be used as medical devices suitable for replacing toxic chemicals, disinfectants and preservatives.

### BACKGROUND OF THE INVENTION

1. Technical Field

The present invention relates to saving and keeping things from decaying, decomposition, deterioration, spoiling, putrefication or fermentation through the safe reduction or elimination of the natural carious processes which result in unwanted infectious causing or contaminating chemical changes or in the growth of pathogenic bacteria or viruses. This invention avoids hazardous toxic chemicals, preservatives and embalming substances with safe “green-type” risk-free harmless specially formulated non-toxic, odorless, inexpensive, nano-scopic liquid preservative formulated solutions and embalming medical devices that have superior preservation, anti-microbial, decontaminating and embalming properties.

2. History Preceding the Invention

Modern scientists are still trying to uncover the secrets of some of the ancient Old World preservation technologies for mummification that come from Egypt, China and Peru. These included resins, essential oils and numerous other secret embalming procedures that still baffle researchers. It is possible that some of these ancient embalming artists knew about and took advantage of the widely known anti-microbial, preservation, anti-pathogenic and safe-guarding properties of the precious mineral known as silver. This precious metal has been in use since 4,000 BC (as a preservative and for a wide variety of healing purposes as well as for treating diseases and wounds). Eastern Persian records specifically mention the practice of placing water in silver vessels, and the ancient Babylonian and Greek civilizations were fully aware of silver's ability to preserve and disinfect.

According to the Greek historian, Herodotus, called the “Father of History”, Persian kings, including Cyrus (who conquered Babylon), would only drink water from their own Choaspes River that flowed past the ancient capital of Susa. Wherever the kings went, a long train of four-wheeled mule wagons followed them transporting silver jars filled with boiled water from the river's waters. The water would remain fresh for years during the long campaigns.

At the height of the Roman Empire, silver compounds were used for medical treatment. During both the Dark and Middle Ages, silver (in one form or another) was used to disinfect open wounds and keep both food and water from becoming contaminated by the Egyptians, Greeks, Romans and Phoenicians. Silver plates, bowls, utensils and silverware protected many of the wealthy by warding off diseases during the infamous plagues and pandemics. Members of royalty were often called “bluebloods,” probably due to the silver content in their blood and the blueness of their skin. When eating, the royalty used almost exclusively silver plates, bowls, utensils and tableware. They essentially digested large particles of silver or silver oxides over an extended period of time, which likely created a permanent blue-grey discoloration of the skin, possibly due to the excessive silver deposits. Many of the pioneers of the “Old West” used silverware plus coins in their containers to prevent spoilage of their milk and water.

In the last century, scientists were beginning to develop more sophisticated forms of silver to kill germs. The USFDA has been regulating the uses of silver since 1913. In 1924, the first electro-colloidal silver was produced, resulting in tiny particles with positive charges that stayed in suspension. In March of 1978, Science Digest called colloidal silver the mightiest germ fighter known to medicine. The use of silver preparations in modern, conventional medicine has survived until this Modern Age. Among them are the uses of dilute silver nitrates in newborn babies' eyes to protect the infants from infection. Another important use is a silver based salve (called “Silvadine”) that can be found in virtually every burn ward in North America to fight infection. Additionally, a patented silver coated nylon material (known as “Silvalon”) was licensed by the FDA as an anti-microbial bandage. Clearly, silver has historically been one of mankind's most reliable tools in preserving foods and water, in retarding infections and in supporting the immune system against various diseases, even before humans knew what caused these maladies. After reviewing 23 different water purification and preservation systems, NASA selected a silver-based system for the space shuttle, and Japan uses silver air purifiers. Ayurvedic physicians of India continue to use it for medicinal purposes to this day. Since the introduction of patented antibiotics for medicinal uses by the medical industries in the 1930s, the pharmaceutical industry has dominated anti-microbial science. Since that time, silver preparations and solutions have dramatically receded from general utilization until recently.

3. Research Behind this Invention

The combined expertise and backgrounds and relationships to any form or type of silver applications of the inventors of this invention include: actuarial sciences, medical research/distribution and sales/supply, industrial technologies, archaeology, anthropology, scientific wellness/optimal health and longevity investigations, infection control and sterile processing and waste management spanning over up to four decades.

Many years ago, one of the inventors began independently investigating a variety of experiments evaluating the preservative applications of ionized colloidal silver in non-toxic, non-hazardous waste, safe “green-type” anti-microbial systems and methods for: (1) healthful preservation of foods and drinks (without latent chemical side-effects), (2) embalming (of bodies, organs and tissues); (3) preventing unwanted bacterial or viral growth in a wide variety of applications; (4) retarding the natural decaying processes in foods and beverages; and, (5) preserving and safe-guarding against the customary exponential growth of pathogenic organisms in either sterile and/or polluted environments.

It was noticed that the spraying of non-toxic, safe “green-type” small colloidal silver particles in suspension in pure water solutions appeared to have some of the same preservative characteristics similar to standard chemical but toxic preservatives. It was repeatedly observed that a wide variety of foods lasted longer without the usual spoilage when sprayed or soaked in these non-toxic, safe “green-type” small colloidal silver particles in suspension in pure water solutions. It was also discovered that the longevity and usefulness of laboratory research specimens was greatly improved by similar exposure to the same non-toxic, safe “green-type” small colloidal silver particles in suspension in pure water solutions. Wanting more information, this inventor next supervised the precise application of specially formulated solutions of nano-sized colloidal particles of pure silver to various “killer molds and fungi” commonly found in dark places in the moist and tropical Eastern and Western Coastal Areas of North, Central and South America. The lethal results of controlled applications of these specially formulated suspended nano-sized particles of pure silver solutions to these highly toxic molds and fungi was successful, and the inventor initiated some further research in controlled laboratory tests on the application of various concentrations of nano-sized colloidal particles of pure silver in stable solutions to selected pathogenic bacterial and viral organisms.

During the next few years, numerous private clinically controlled laboratory and field tests were done to determine the optimal PPM (parts per million concentrations) of nano-sized colloidal particles of pure silver for the best kill ratio when applied to these pathogenic organisms. Between 1996 and 2004, the research was expanded with the help of Joseph Goodman, Ph.D., a highly published pioneering electron-microscope scientist at the University of California at San Francisco. These electron-microscopy studies focused on micro as well as nano-sized colloidal particles of pure silver. The purpose of this project utilizing electron-microscopy research was to study the processes and effects of both positively charged micro-sized (500-900 microns) as well as much smaller minute (10-20 microns) nano-sized colloidal particles of pure silver on extremely toxic and pathogenic bacteria. A series of research phases was launched to investigate the annihilation of pathogenic bacteria by colloidal silver.

The first phase centered around the analyzing of the efficiency of attachment to pathogenic cells between the large micro-sized (which often resulted in silver clumps) and the very minute non-clumping (permanently stable) suspended nano-sized colloidal particles of pure silver. The study of particle size clearly demonstrated that the smallest non-clumping nano-sized colloidal particles of pure silver were far superior to the larger micro-sized molecules in remaining attached to the outside cell walls of the pathogenic bacteria organisms.

The second phase analyzed the penetration of the bacteria's cell membrane wall by both the large micro-sized (which often resulted in silver clumps that were unable to penetrate cell walls) and the very minute non-clumping (permanently stable) suspended nano-sized colloidal particles of pure silver. For the most efficient cell wall penetration, it became evident that the silver particle size is extremely important for successful invasion of the bacteria cell wall. Large clumps of colloidal silver rarely penetrated even the thinnest or weakest sections of the bacteria cell walls. In the rare cases where micro-sized clumps of silver invaded the cell, they almost never attached to the cell nucleus. The limited successes in annihilating pathogenic bacteria by the larger micro-sized clumps of silver caused the researchers to discontinue the study of the larger silver colloidal particles in the next phases of the electron-microscopy research.

The third phase investigated the genocidal behaviors of the minute nano-sized colloidal particles of pure silver inside the cell organism. Of particular interest was the consistent attachment to the core nucleus of the bacteria cell by the positively charged nano-sized colloidal silver particles.

The fourth phase of the research centered on the phenomenal implosion and ruination of the diseased or pathogenic cells into small protein fragments soon after the positive nano-sized colloidal silver particle became bonded with the negatively charged nucleus. This complete destructive fragmentation into harmless protein particles of the pathogenic cell was observed repeatedly soon after the nano-sized colloidal particles of pure silver attached to the nucleus. This disintegration mechanism appeared similar in process to the lethal actions and processes of the Immunocompetent white blood cells known as “NK cells” that have been observed invading and imploding targeted pathogenic organisms in the vicinity of the lymphocytic “NK cell.”

It was noted with great interest throughout these years of electron-microscopy research that the smaller and minute nano-sized colloidal particles of pure silver destroyed all anaerobic bacterial and viral cells that were tested. These included many pathogenic organisms, such as:
E. coli, food poisoning, ordinary tract infections, traveler's diarrhea, infantile diarrhea, respiratory tract infections
Enterobacter cloace (produces illnesses similar to the E. aerogenes)
Klebsiella pneumoniae, Nosocomial infections (the ones that spread through hospitals), lower respiratory tract infections, bacteremia, and urinary tract and wound infections
Klebsiella oxytoca, Produces illnesses similar to those infectious conditions caused by K. pneumoniae
Pseudomonas aerugio/nosa, keratitis, pneumonia, meningitis, severe burn and wound infections, nosocomial infections, urinary tract infections, etc.
Streptococcus pyogenes, skin infections, upper respiratory infections (such as strep throat), impetigo, scarlet fever, hospital-acquired infections, etc.
Streptococcus faecalis, urinary tract infections, endocarditis, wound infections, etc.
Streptococcus mutans (a major cause of dental plaque and tooth decay)
Streptococcus gordonii, tooth decay, and also implicated in infective endocarditis (an infection of the heart valves)

Various medical journals have also documented the successful uses of Colloidal Silver against the following pathogenic organisms: Anthrax bacilli, B. coli, B. coli communis, B. dysenteria, B. tuberculosis, Gonococcus, Paramecium, Pneumococci, Straphyloclysin, Straphiococcus pyogens albus, Straphiococcus pyogens aureus, Tinea versicolor, Vortic Aspergillus niger, Bacillus typhosus, Bovine Rotavirus, Candida albicans, Legionella pneumophilia, Poliovirus I1 (Sabin Strain), Salmonella, Vegetative B. cereus spores and Spore-Forming Bacteria.

In the late 1960s, one of the inventors developed a silver recovery system in the x-ray field when he discovered that there was about one oz. of silver per gallon that was processed into waste after every piece of chest x-ray size film developed, but the cost of recovery exceeded the price of silver at that time. H is next experience with the preservation and healing properties of silver came in the late 1970s when he suffered serious and potentially debilitating burns to an arm from his finger nails all the way to the mid-forearm. These burns were 2^{nd} degree and suppose to leave bad scars. The pigment in the skin had turned white plus there were serious physical limitations to the movement of his fingers and wrist. Thanks to his doctor who had just learned about a new silver-containing product released to the military to treat severe burns, every day for around 21 days he would go to the doctor's office for medical debridement on the decayed and dead skin by completely swabbing the burned area with the product. The silver treatment was 100% successful in saving and preventing any damage, temporary or permanent, to his hand and forearm. Today, there is no evidence of the burned skin.

Additionally, this inventor has worked as an expert in infection control and sterile processing as a manufacturer's certified teaching in service representative specializing in all phases of infection control and sterile processing, infection policies and procedures, inspection protocols, contamination and cross contamination, re-infection, re-contamination and all aspects of sterile processing. He focused mainly on helping various departments obtain national certification from agencies inspecting hospitals every few years, as well as including all areas of the healthcare industry to include human, dental, non-hospital, veterinary, and open areas of the medical industry, such as blood banks, etc. During these same years, he also specialized in processing medical waste sterilization and disposal (without burning it) in order to make it a (1) harmless substance, and (2) get rid of it, by shredding it (so that nothing whole went to a landfill) with zero pollution to the land, water or air.

### 4. Description and Analyses of Prior Art

There are no exact uses or previous applications envisioned that might be possible, even with drastic modifications, in any of the prior art described or analyzed herein. Thus, none of the prior art relates in any manner whatsoever to the unique intended uses and applications of this invention.

U.S. Pat. No. 5,326,567 is directed to an anti-microbial metal-based composition with silver as one of several composites with limited applications and specified infection-resistance uses as topical coatings for treatment of infections caused by bacteria, fungus and viruses in humans and animals. The compositions can also be used for treating medical devices and adhesives.

U.S. Pat. No. 5,464,559 is directed to a composition product with limited applications and specified uses for treating and disinfecting drinking water by using the method of U.S. Pat. No. 2,434,190 to produce a gel form or resin salt based form of 0.5 mol silver ions in order to remove iodide and disinfect drinking water. The primary and limited uses of this patent are substantially different in all respects from the present invention.

U.S. Pat. No. 6,027,469 is directed to a limited disinfectant application with specific uses for disinfecting dialysis equipment only. Even though this application utilizes ionized particles of silver, they tend to clump, will not mix, and otherwise “fall out of suspension” when (A) not manufactured small enough; (B) manufactured in too high of concentrations, or when they come in proximity with any other “Anti” charge elements including any metal.

U.S. Pat. No. 6,093,414 is directed to a silver-based anti-microbial composition which relates to the processes of making several composites that are useful in the treatment and prevention of infections and diseases.

U.S. Pat. Nos. 6,583,176 and 7,261,905 relate to the making and limited applications and specified uses of aqueous liquid disinfectant solutions for specified uses as prevention against contamination by potentially pathogenic bacteria and viruses. The main difference between the two patents is that one product is made from a solution of silver ion and an acid complex instead of a citric complex. The acid in this formula is an organic acid and then combined with ionic silver and water. Otherwise the products in the patents are the same and the uses are the same.

U.S. Pat. No. 7,147,845 refers to coated or impregnated anti-microbial silver ion complex resinate compositions that can be loaded onto an anion exchange resin. This resinate composition has antiviral or antifungal activity as a dressing such as a bandage or wound pad that may be useful in the treatment and prevention of infections and diseases.

### SUMMARY OF THE INVENTION

The invention is directed to a method of preparing and producing non-toxic liquid preservative substance solutions that can be utilized as embalming solutions, sterilizing agent solutions, disinfectant solutions, preservative solutions, mold genocidal solutions and fungus extermination solutions in order to forestall or prevent unwanted chemical and biological changes or microbial/bacterial actions plus retard deterioration and decomposition or other negative changes. The method comprises the steps of first creating at least one scheme for the preparation and production of the non-toxic liquid solutions. Then, the scheme is used to prepare and produce a formulated solution composed of extremely minute nano-sized particles of 99.99% pure colloidal silver in a concentration ranging from 2-80 ppm permanently suspended in a pure distilled water aqueous solution. The formulated solution is then checked and evaluated for quality control, and, finally, the solution is prepared for shipment and delivery.

### BRIEF DESCRIPTION OF DRAWINGS

The invention is described in greater detail in the following description of examples embodying the best mode of the invention, taken in conjunction with the drawing figures, in which:

FIG. 1 is a block diagram illustrating a preferred form of the invention.

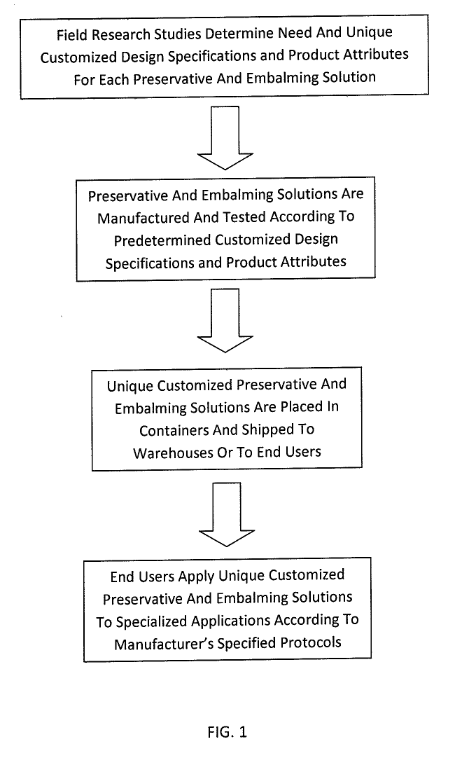

FIG. 2 is a schematic diagram showing the methodologies of the customized manufacturing processes of the methods according to the invention and the procedural protocols of the various design aspects of the invention.

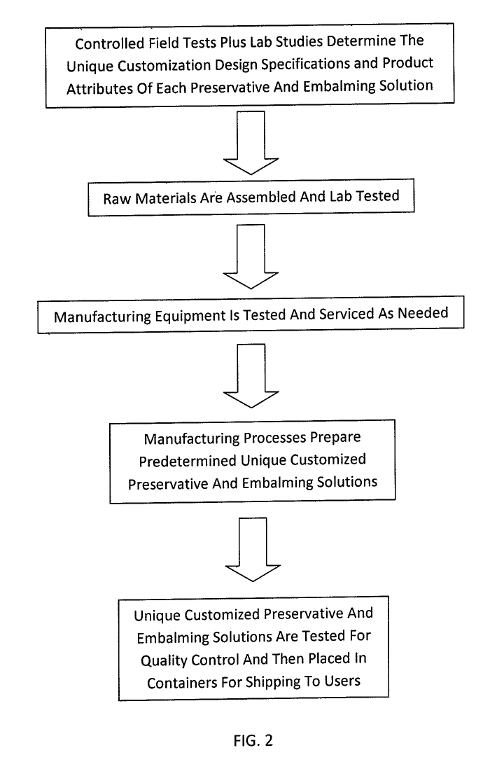

FIG. 3 is a schematic diagram showing the application of the system according to the invention and the procedural protocols of the various facets of the invention.

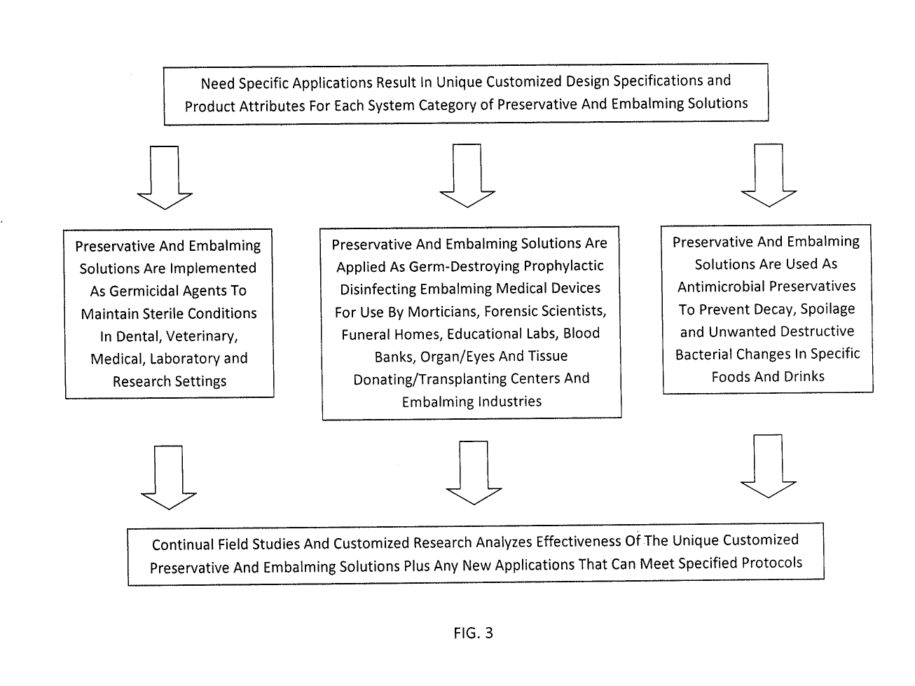

### DESCRIPTION OF EXAMPLES EMBODYING THE BEST MODE OF THE INVENTION

The invention is directed to use of single nano-sized metallic elements in totally sterile, water based solutions. It utilizes stand-alone, nano-sized silver ionic particles that can attach to a carrier (like a water particle), travel over a distance (naturally or by force), and then engage and destroy on contact all forms of pathogenic organisms that may cause decay, decomposition, deterioration and/or disease in either humans or animals. It is stable with a long shelf life.

The invention can be widely utilized as a safe, non-toxic ionized silver particle (nano-sized) processed mineral water of maximum purity that is useful as a preservative. This capability creates the conditions desirable for preventing decay, and therefore makes for a desirable preservative. The present invention is very amenable to I.V. injection and can therefore be used in a wide variety of ways. Some examples are: (1) Human cadavers (embalming) as well as preservation of deceased animals. (2) Customized I.V. injection into blood bags to prevent contamination and preserve whole blood. (3) Any intravenous injection for multiple purposes.

The present uses pure silver as the only active ingredient and its stand alone proven killing abilities against pathogenic organisms that may cause decay, decomposition, deterioration and/or disease. All other patents use silver as an ingredient combined with other ingredients. The prior art depends on the total combined killing efficacy of all ingredients after they are combined into a composition. It is unlikely that any of the prior art any preservation application capabilities or stand-alone pathogenic killing efficacies because of their combinations with other ingredients.

The present invention consists of unique application methodologies and modes for preparing and producing as well as complete customized systems for implementing and utilizing safe “green-type” non-toxic liquid specific formulated nano-sized colloidal silver non-clumping preservative substance solutions that can be utilized as organic solutions to preserve sterile conditions and forestall or prevent unwanted infectious causing or contaminating chemical and biological changes or microbial/bacterial actions, plus retard deterioration and decomposition or unacceptable negative changes. The formulated customized preservative applications are designed to be used as embalming medical devices that can significantly retard the carious destructive processes of nature and have anti-microbial properties. The preservative applications are comprised of specially formulated minute nano-sized colloidal silver particles in sterile liquefied stable suspended preparations. Additionally, these specific formulated customized preservative applications may be added (as a healthy safe non-toxic preservative) to food and/or drink products. These specially formulated customized nano-scopic mixtures contain electrically charged nano-particle ions of pure silver in complete stable suspension and uniform steady-state non-clumping formulations. The invention provides precise formulations and protocols of manufacturing processes that will result in controlled ultra-refined nano-sized uniform silver ionic particles necessary for liquid solutions to be used as: (1) anti-microbial and germicidal agents for the preservation of sterile conditions in dental, veterinary, medical, laboratory and research and any other settings requiring the preservation of sterile conditions; (2) germ-destroying prophylactic disinfecting embalming medical devices suitable for risk-free and harmless solutions for use by morticians, forensic scientists, funeral homes, educational and industrial/research laboratories, blood banks, organ/eyes and tissue donating/transplanting centers and the embalming industries; and, (2) anti-microbial preservatives for the prevention of decay and/or bacterial changes in foods and drinks.

The present invention provides comprehensive schemes and procedures for the preparation, production and customized applications which utilize proprietary safe “green-type” non-toxic liquid formulated nano-sized colloidal silver non-clumping preservative substance solutions which result in (1) the preservation of desirable sterile conditions; or, (2) the retardation of expected natural carious processes; or, (3) the reduction or eliminating of negative changes or undesired microbial and bacterial actions; or, (4) the forestalling of expected biological deterioration, decomposition, putrefication, spoiling, decay or fermentation; or, (5) the prevention of unwanted infectious causing or contaminating chemical changes; and/or, (6) the preservation of food and drink products. The specific prepared preservative customized applications are designed to be utilized in preserving desirable sterile conditions as well as be used as embalming medical devices that can significantly retard the carious destructive processes of nature as well as have positive anti-microbial properties comprised of specially formulated customized minute nano-sized colloidal silver particles in sterile liquefied stable suspended preparations. These specially formulated custom-made nano-scopic mixtures contain electrically charged nano-particle ions of pure colloidal silver with a fineness of 0.999+ purity in complete stable suspension and uniform steady-state non-clumping customized formulations. The preservation methods and systems take advantage of existing technology based equipment by utilizing customized design procedures, precise formulations and protocols of custom-made manufacturing processes that result in controlled ultra-refined nano-sized uniform silver ionic particles necessary for the specially formulated proprietary customized liquid solutions that can also be used as: (1) germicidal agents for the preservation of desirable sterile conditions in dental, veterinary, medical, laboratory and research and any other settings requiring the maintenance of sterile conditions; or, (2) germ-destroying prophylactic disinfecting embalming medical devices suitable for replacing toxic chemicals with risk-free and harmless solutions for use by morticians, forensic scientists, funeral homes, educational laboratories, blood banks, organ/eyes and tissue donating/transplanting centers and the embalming industries; or, (3) anti-microbial preservatives for the prevention of unwelcomed decay and negative or pathogenic bacterial changes in both foods and/or drinks.

In accordance with the present invention, the water utilized must be pure. The distilled water aqueous solution has less than 25 ppm (parts per million) inert dissolved solids, with no toxic substances whatsoever included. The nano-sized particles of pure colloidal silver are about 1-5 nanometers in size. The silver is at least 99.99% pure.

In one form of the method, non-toxic liquid preservative substance solutions are prepared that can be utilized as embalming solutions in order to forestall or prevent unwanted chemical and biological changes or microbial/bacterial actions, plus retired deterioration and decomposition or other negative changes. The method comprises the steps of first creating at least one scheme for the preparation and production of the non-toxic liquid solutions for an embalming application. Then, the scheme is utilized to prepare and produce formulated embalming solutions composed of extremely minute nano-sized particles of pure colloidal silver in a concentration ranging from 2-80 ppm permanently suspended in a pure distilled water aqueous solution. Quality control occurs next, checking and evaluating the formulated embalming solutions and, finally, the formulated embalming solutions are prepared for shipment and delivery.

The solutions can be used as germ-destroying prophylactic embalming medical supplies for utilization and applications in:
a) mortuaries and funeral homes;
b) coroners and morticians;
c) forensic scientific offices and research laboratories;
d) educational and research laboratories;
e) organ/eyes and tissue donating/transplanting centers;
f) embalming companies and industrial units; and
g) any other applications requiring germ-destroying, prophylactic, embalming medical devices.

The same method is used for preparing and producing non-toxic liquid preservative substance solutions that can be utilized as sterilizing agent solutions. In this form of the method, the formulated sterilizing solutions can be used as germ-destroying prophylactic medical sterilization devices in:
a) hospitals, community clinics, hospices and nursing facilities;
b) animal husbandry, veterinary offices and laboratories;
c) medical and dental offices and laboratories;
d) optometry offices and laboratories;
e) forensic science centers and laboratories;
f) blood banks, compounding pharmacies;
g) out-patient, surgery and patient care centers;
h) assisted living and retirement communities;
i) mobile diagnostic and medical care units;
j) surgery, emergency care centers and medical clinics;
k) public health, police, fire, emergency and Governmental Bodies;
l) military, Homeland Security and National Defense Units;
m) medical, scientific laboratories and research facilities; and
n) any other settings (medical, non-medical, human, non-human) requiring preservation of sterile conditions.

The same method can also be used in preparing and producing non-toxic liquid preservative substance solutions that can be utilized as disinfectant solutions. In this form of the invention, the disinfectant solutions can be used as germ-destroying prophylactic disinfecting medical devices in:
a) office buildings and laboratories,
b) commercial buildings and warehouses;
c) educational settings and gymnasiums;
d) schools, libraries and churches;
e) retail businesses and restaurants;
f) agricultural and farming organizations;
g) public service and private institutions,
h) churches, educational schools and public libraries;
i) apartments, residential and commercial buildings;
j) buses, trains, airplanes, automobiles and trucks;
k) swimming pools, salons and spas;
l) country clubs, resorts and hotels;
m) mechanic shops and other repair settings;
n) buildings or structures with mold and/or fungi infestations; and
o) any other places requiring disinfecting treatments for toxic conditions.

Also, the same process can be used for preparing non-toxic liquid preservative substance solutions that can be utilized as preservative solutions. In this form of the invention, the preservative solutions can be used in:
a) industrial or commercial uses of non-toxic preservatives, including but not limited to paints, glues, carpets;
b) other residential, commercial or industrial settings requiring non-toxic preservatives or chemicals;
c) sprays, environmental and washing applications for the preservation of foods; and
d) selected liquid/solid foods and drinks.

The invention can also be used in producing non-toxic liquid preservative substance solutions that can be utilized as mold genocidal solutions. In this form of the invention, the mold genocidal solutions can be used in:
a) office buildings and laboratories,
b) commercial buildings and warehouses;
c) educational settings and gymnasiums;
d) schools, libraries and churches;
e) retail businesses and restaurants;
f) agricultural and farming organizations;
g) public service and private institutions,
h) churches, educational schools and public libraries;
i) apartments, residential and commercial buildings;
j) buses, trains, airplanes, automobiles and trucks;
k) swimming pools, salons and spas;
l) country clubs, resorts and hotels;
m) mechanic shops and other repair settings;
n) buildings or structures with pathogenic or deadly mold; and
o) any other places requiring non-toxic mold exterminating treatments for toxic conditions.

Finally, the method can be used in producing non-toxic liquid preservative substance solutions that can be utilized as fungus extermination solutions. In this form of the invention, the fungus extermination solutions can be used in:
a) office buildings and laboratories,
b) commercial buildings and warehouses;
c) educational settings and gymnasiums;
d) schools, libraries and churches;
e) retail businesses and restaurants;
f) agricultural and farming organizations;
g) public service and private institutions,
h) churches, educational schools and public libraries;
i) apartments, residential and commercial buildings;
j) buses, trains, airplanes, automobiles and trucks;
k) swimming pools, salons and spas;
l) country clubs, resorts and hotels;
m) mechanic shops and other repair settings;
n) buildings or structures with pathogenic or deadly fungi infestations; and
o) any other places requiring non-toxic fungi eliminating treatments for toxic conditions.

Various changes can be made to the invention without departing from the spirit thereof or scope of the following claims.

What is claimed is:

1. A method of preparing and producing non-toxic liquid preservative substance solutions that can be utilized as embalming solutions in order to forestall or prevent unwanted chemical and biological changes or microbial/bacterial actions, plus retard deterioration and decomposition or other negative changes, the method comprising the steps of:
a) creating at least one scheme for the preparation and production of the non-toxic liquid solutions for an embalming application;
b) utilizing the scheme to prepare and produce formulated embalming solutions composed of extremely minute nano-sized particles of 99.99% pure colloidal silver in a concentration ranging from 2 to 50 ppm (parts per million) permanently suspended in a pure distilled water aqueous solution;
c) checking and evaluating the formulated embalming solutions for quality control; and
d) preparing the formulated embalming solutions for shipment and delivery.

2. The method according to claim 1 including establishing customized applications of the formulated embalming solutions that can be used as germ-destroying prophylactic embalming medical devices for utilization and applications in:
a) mortuaries and funeral homes;
b) coroners and morticians;
c) forensic scientific offices and research laboratories;
d) educational and research laboratories;
e) organ/eyes and tissue donating/transplanting centers;
f) embalming companies and industrial units; and
g) other applications requiring germ-destroying, prophylactic, embalming medical devices.

3. A method of preparing and producing non-toxic liquid preservative substance solutions that can be utilized as sterilizing agent solutions in order to maintain sterile conditions and forestall or prevent unwanted environmental chemical and biological changes or microbial/bacterial actions or other negative changes, the method comprising the steps of:
a) creating at least one scheme for the preparation and production of the non-toxic liquid solution for a sterilization application;
b) utilizing the scheme to prepare and produce formulated sterilizing solutions composed of extremely minute nano-sized particles of 99.99% pure colloidal silver in a concentration ranging from 2 to 50 ppm (parts per million) permanently suspended in a pure distilled water aqueous solution;
c) checking and evaluating the formulated sterilizing solutions for quality control; and
d) preparing the formulated sterilizing solutions for shipment and delivery.

4. The method according to claim 3 including establishing customized applications of the formulated sterilizing solutions that can be used as germ-destroying prophylactic medical sterilization devices for utilization and applications in:
a) hospitals, community clinics, hospices and nursing facilities;
b) animal husbandry, veterinary offices and laboratories;
c) medical and dental offices and laboratories;
d) optometry offices and laboratories;
e) forensic science centers and laboratories;
f) blood banks, compounding pharmacies;
g) out-patient, surgery and patient care centers;
h) assisted living and retirement communities;
i) mobile diagnostic and medical care units;
j) surgery, emergency care centers and medical clinics;
k) public health, police, fire, emergency and Governmental Bodies;
l) military, Homeland Security and National Defense Units;
m) medical, scientific laboratories and research facilities; and
n) other settings (medical, non-medical, human, non-human) requiring preservation of sterile conditions.

5. A method of preparing and producing non-toxic liquid preservative substance solutions that can be utilized as disinfectant solutions in order to reduce or eliminate pathogenic bacterial and viral growths and forestall or prevent unwanted environmental chemical and biological changes or microbial/bacterial actions or other negative changes, the method comprising the steps of:
a) creating at least one scheme for the preparation and production of the non-toxic liquid solution for a disinfecting application;
b) utilizing the scheme to prepare and produce formulated disinfectant solutions composed of extremely minute nano-sized particles of 99.99% pure colloidal silver in a concentration ranging from 2 to 50 ppm (parts per million) permanently suspended in a pure distilled water aqueous solution;
c) checking and evaluating the formulated disinfectant solutions for quality control; and
d) preparing the formulated disinfectant solutions for shipment and delivery.

6. The method according to claim 5 including establishing customized applications of the formulated disinfectant solutions that can be used as germ-destroying prophylactic disinfecting medical devices for utilization and applications in:
a) office buildings and laboratories,
b) commercial buildings and warehouses;
c) educational settings and gymnasiums;
d) schools, libraries and churches;
e) retail businesses and restaurants;
f) agricultural and farming organizations;
g) public service and private institutions,
h) churches, educational schools and public libraries;
i) apartments, residential and commercial buildings;
j) buses, trains, airplanes, automobiles and trucks;
k) swimming pools, salons and spas;
l) country clubs, resorts and hotels;
m) mechanic shops and other repair settings;
n) buildings or structures with mold and/or fungi infestations; and
o) other places requiring disinfecting treatments for toxic conditions.

7. A method of preparing and producing non-toxic liquid preservative substance solutions that can be utilized as preservative solutions in order to reduce or eliminate pathogenic bacterial and viral growths and forestall or prevent unwanted environmental chemical and biological changes or microbial/bacterial actions or other negative changes, the method comprising the steps of:
a) creating at least one scheme for the preparation and production of the non-toxic liquid solution for a preservative application;
b) utilizing the scheme to prepare and produce formulated preservative solutions composed of extremely minute nano-sized particles of 99.99% pure colloidal silver in a concentration ranging from 2 to 50 ppm (parts per million) permanently suspended in a pure distilled water aqueous solution;
c) checking and evaluating the formulated preservative solutions for quality control; and
d) preparing the formulated preservative solutions for shipment and delivery.

8. The method according to claim 7 including establishing customized applications of the formulated preservative solutions that can be used as germ-destroying prophylactic preservative solutions for utilization and applications in:
a) industrial or commercial uses of non-toxic preservatives, including but not limited to paints, glues, carpets;
b) other residential, commercial or industrial settings requiring non-toxic preservatives or chemicals;
c) sprays, environmental and washing applications for the preservation of foods; and
d) selected liquid/solid foods and drinks.

9. A method of preparing and producing non-toxic liquid preservative substance solutions that can be utilized as mold genocidal solutions in order to reduce or eliminate pathogenic mold growths and forestall or prevent unwanted environmental chemical and/or biological changes or microbial/bacterial actions or other negative changes, the method comprising the steps of:
a) creating at least one scheme for the preparation and production of the non-toxic liquid solution for a mold genocidal application;
b) utilizing the scheme to prepare and produce formulated mold genocidal solutions composed of extremely minute nano-sized particles of 99.99% pure colloidal silver in a concentration ranging from 2 to 50 ppm (parts per million) permanently suspended in a pure distilled water aqueous solution;
c) checking and evaluating the formulated mold genocidal solutions for quality control; and
d) preparing the formulated mold genocidal solutions for shipment and delivery.

10. The method according to claim 9 including establishing customized applications of the formulated mold genocidal solutions that can be used as mold-destroying prophylactic extermination devices for utilization and applications in:
a) office buildings and laboratories,
b) commercial buildings and warehouses;
c) educational settings and gymnasiums;
d) schools, libraries and churches;
e) retail businesses and restaurants;
f) agricultural and farming organizations;
g) public service and private institutions,
h) churches, educational schools and public libraries;
i) apartments, residential and commercial buildings;
j) buses, trains, airplanes, automobiles and trucks;
k) swimming pools, salons and spas;
l) country clubs, resorts and hotels;
m) mechanic shops and other repair settings;
n) buildings or structures with pathogenic or deadly mold; and
o) other places requiring non-toxic mold exterminating treatments for toxic conditions.

11. A method of preparing and producing non-toxic liquid preservative substance solutions that can be utilized as fungus extermination solutions in order to reduce or eliminate pathogenic or deadly fungal growths and forestall or prevent unwanted environmental chemical and biological changes or microbial/bacterial actions or other negative changes, the method comprising the steps of:
a) creating at least one scheme for the preparation and production of the non-toxic liquid solution for a fungus extermination application;
b) utilizing the scheme to prepare and produce formulated fungus extermination solutions composed of extremely minute nano-sized particles of 99.99% pure colloidal silver in a concentration ranging from 2 to 50 ppm (parts per million) permanently suspended in a pure distilled water aqueous solution;
c) checking and evaluating the formulated fungus extermination solutions for quality control; and
d) preparing the formulated fungus extermination solutions for shipment and delivery.

12. The method according to claim 9 including establishing customized applications of the formulated fungus extermination solutions that can be used as fungus destroying prophylactic genocidal devices for utilization and applications in:
a) office buildings and laboratories,
b) commercial buildings and warehouses;
c) educational settings and gymnasiums;
d) schools, libraries and churches;
e) retail businesses and restaurants;
f) agricultural and farming organizations;
g) public service and private institutions,
h) churches, educational schools and public libraries;
i) apartments, residential and commercial buildings;
j) buses, trains, airplanes, automobiles and trucks;
k) swimming pools, salons and spas;
l) country clubs, resorts and hotels;
m) mechanic shops and other repair settings;
n) buildings or structures with pathogenic or deadly fungi infestations; and
o) other places requiring non-toxic fungi eliminating treatments for toxic conditions.
